(19) United States
(12) Patent Application Publication
(10) Pub. No.: US 2023/0363923 A1
(43) Pub. Date: Nov. 16, 2023

(54) REVERSIBLY, PROXIMALLY, AND DISTALLY EXPANDABLE SPINAL CAGE

(71) Applicant: Teslake, Inc., San Diego, CA (US)

(72) Inventor: Lukas Eisermann, San Diego, CA (US)

(21) Appl. No.: 17/742,305

(22) Filed: May 11, 2022

(51) Int. Cl. A61F 2/44

(52) U.S. Cl. CPC A61F 2/4455; A61F 2002/4627

(57) ABSTRACT

An implant includes a cage and a slider mechanism. The cage has a cage length, a cage width, proximal height, and distal height. The cage includes an upper support, a lower support a first serpentine spring, and a second serpentine spring. The first serpentine spring joins the upper support to the lower support along a first path having a first path length that is greater than the cage length. The second serpentine spring joins the upper support to the lower support along a second path having a second path length that is greater than the cage length. The slider mechanism includes a proximal slider mechanism and a distal slider mechanism. The proximal slider mechanism is configured to adjust the proximal height of the cage. The distal slider mechanism is configured to adjust the distal height of the cage.

### FIELD OF THE INVENTION

The present disclosure relates to implantable orthopedic devices for stabilizing the spine. More particularly, the present disclosure describes an implant that can be reversibly and variably expanded and contracted.

### BACKGROUND

Implantable devices such as cages and spacers are in use for providing support between sequential vertebrae of a human spine. Such devices are selected to provide a precision fitment for the space between the sequential vertebrae. Matching a device precisely can be challenging due to geometric variation. In addition, there may be a need to remove such a device after they are implanted. There is a need for an expandable cage that can be adapted to a variable geometry including the spacing and angular relationship between surfaces of the sequential vertebrae. Also, there is a need for a cage that can be later removed with minimal effect on an implant site.

### BRIEF DESCRIPTION OF DRAWINGS

FIG. 1 is an isometric drawing of a first embodiment of an implant having a cage and a slider mechanism for altering an outer geometry of the cage.

FIG. 2 is a side view of a first embodiment of an implant.

FIG. 3 is an isometric bi-cross sectional view of a first embodiment of an implant.

FIG. 4 is a side bisectional view of a first embodiment of an implant.

FIG. 5 is an isometric view of an implant mounted to an insertion instrument.

FIG. 6 is a side bisectional view of an implant mounted to an insertion instrument.

FIG. 6A is detail taken from FIG. 6.

FIG. 7 is a flowchart depicting a method of mounting an implant at a surgical site.

FIG. 8 is an isometric view of a second embodiment of an implant.

FIG. 9 is a side bisectional view of a second embodiment of an implant.

FIG. 10 is an isometric view of a third embodiment of an implant.

FIG. 11 is a top sectional view of a third embodiment of an implant.

FIG. 12 is a side view of a third embodiment of an implant.

FIG. 13 is a vertical side cross sectional view of a third embodiment of an implant bisecting the third and fourth quadrants shown in FIG. 11.

### SUMMARY

The disclosure that follows infra describes an implant having an outer cage and a slider mechanism for altering an outer geometry of the outer cage. The cage has proximal and distal ends with respect to a major axis of the cage. The slider mechanism is configured to independently adjust a proximal and distal height of the cage at the proximal and distal ends respectively. The cage in combination with the slider mechanism is configured to provide reversible expansion and contraction of the cage without hysteresis effects by operating within elastic limits of the cage.

In a first aspect of the disclosure, an implant includes a cage and a slider mechanism. The cage refers to the outer housing of the implant, and has a cage length, a cage width, proximal height, and distal height. The cage length is along a major axis X of the cage between a proximal end and a distal end of the cage. The cage width is a long a lateral axis Y. The proximal height is a vertical height of the proximal end of the cage. The distal height is a vertical height of the distal end of the cage. The cage includes an upper support, a lower support, a first serpentine spring, and a second serpentine spring. The first serpentine spring joins the upper support to the lower support along a first path having a first path length that is greater than the cage length. The first path generally lies along a first side of the cage. The second serpentine spring joins the upper support to the lower support along a second path having a second path length that is greater than the cage length. The second path generally lies along a second side of the cage. The slider mechanism includes a proximal slider mechanism and a distal slider mechanism. The proximal slider mechanism is configured to adjust the proximal height of the cage along a vertical axis Z. The distal slider mechanism is configured to adjust the distal height of the cage along the vertical axis Z. During adjustments of proximal and distal height (increasing and decreasing the height), the serpentine springs remain within their elastic strain limits. This allows for complete reversibility of cage expansion and contraction without hysteresis. This in turn allows the implant to be removed from a surgical site with minimal adverse effects upon surrounding tissue and bone. Axes X, Y and Z are mutually orthogonal.

The “serpentine spring” refers to a spring having a serpentine path geometry. A serpentine path geometry according to the present disclosure is defined along the first and second sides of the cage. The first and second sides of the cage are rectangular sides that are each defined along the X and Z axes. The serpentine path defines a plurality of straight segments that are each parallel to the X axis but arranged or disposed along the Z axis. The straight segments have ends that are joined together with curved or U-shaped portions in an alternating manner in order to define a continuous serpentine path along the serpentine spring from the upper support to the lower support. In an illustrative embodiment, the cage has a cage length L. The length of the serpentine spring measured along the serpentine path is preferably about 3×L or 3L. The serpentine spring has a width and thickness that are each preferably less than 0.05L. Thus, the serpentine spring length measured along the serpentine path is at least 60 times the width or thickness of the spring. These dimensional comparisons can vary by design while allowing the implant and serpentine springs to operate within their elastic strain limits.

The first and second serpentine path lengths are each preferably at least two times the cage length. In some embodiments the first and second path lengths each be at least about 2.5 or 2.8 times the cage length. Longer path lengths are advantageous to minimize strain elongation of the serpentine springs as the cage height is varied so that the serpentine springs remain within their elastic limits when the height of the cage is adjusted. In an illustrative embodiment, the first and second serpentine springs each define two U-shaped bends that connect three linear segments. Each linear segment defines a length greater than 80% of the cage length. This geometry is advantageous for keeping strain within the elastic limit.

The “elastic stress or strain limit” is defined as a threshold of stress or strain above which a object is plastically deformed. Above the elastic strain limit, the object exhibits hysteresis in the stress strain curve. The elastic stress limit is also known as the “yield strength”. The design in the present disclosure allows a stress applied by the slider mechanisms to be below the yield strength of the serpentine springs.

In one implementation the proximal slider mechanism has a proximal height range of adjustment. The distal slider mechanism has a distal height range of adjustment. The first and second serpentine springs are each configured to remain within an elastic strain limit throughout the proximal height range of adjustment and the distal height range of adjustment. Thus, the stress applied to the serpentine springs is below the yield strength.

In another implementation the upper support has a lower proximal surface and a lower distal surface. The lower support has an upper proximal surface and an upper distal surface. The lower and upper proximal surfaces define a taper. The proximal slider mechanism includes a proximal threaded bolt coupled to a proximal slide. Rotating the proximal threaded bolt causes sliding engagement between the proximal slide and the taper defined by the upper and lower proximal surfaces which adjusts a vertical distance between the proximal end of the upper and lower supports. In a similar manner, the lower and upper distal surfaces define a taper. Rotating the distal threaded bolt causes sliding engagement between the distal slide and the taper which adjusts a vertical distance between the distal end of the upper and lower supports.

In yet another implementation the (proximal and/or distal) slider mechanism includes a threaded bolt that is threaded to a slide. The slider mechanism includes a linkage that is rotatively coupled between the slide and the upper support. Rotating the threaded bolt translates the slide which in turn rotates the linkage to increase or decrease a distance between the upper support and the lower support (at the proximal and/or distal end of the cage).

In a further implementation a plurality of tapered locking features extend vertically upward and downward from the upper and lower supports respectively.

In a yet further implementation a plurality of bone screws extend through the upper and lower supports.

In another implementation the proximal slider mechanism includes two side-by-side proximal slider mechanisms. The distal slider mechanism includes two side-by-side distal slider mechanisms. Thus there are four slider mechanisms that allow the height of the cage to be adjusted at four quadrants.

In a second aspect of the disclosure, a system is provided including the implant of the first aspect of the disclosure plus an insertion instrument. The insertion instrument is configured to be inserted through the proximal threaded bolt and to simultaneously couple to the proximal threaded bolt and the distal threaded bolt. This allows both independent or simultaneous adjustment of the vertical distance between the upper and lower supports at both the proximal and distal ends with a single coupling of the insertion instrument to the implant.

In a third aspect of the disclosure, a method for inserting and adjusting an implant is provided for the implant of the first aspect of the disclosure. The method includes coupling the implant upon an insertion instrument, manipulating the insertion instrument to position the implant within a surgical site, operating independent proximal and distal drives to independently adjust the proximal and distal heights of the cage, and decoupling the implant from the insertion instrument.

In one implementation the insertion instrument includes a sleeve coupled to a clamp. Coupling the insertion instrument to the implant includes rotating the sleeve in a first direction to close the clamp over the implant. Decoupling the insertion instrument from the implant includes rotating the sleeve in a second direction that is opposite to the first direction to open the clamp.

In another implementation, operating the independent proximal and distal drives includes independently rotating the proximal and distal drives.

In a fourth aspect of the invention, an implant includes a cage, an upper support, a lower support, a first serpentine spring, a second serpentine spring, and at least two independent slider mechanisms configured to at least adjust the proximal height and the distal height of the cage. The cage has a cage length along a major axis X which is the major axis of the cage. The major axis of the cage extends between a proximal and distal end of the cage. The cage has a cage width along a lateral axis Y from a first side of the cage to a second side of the cage. The cage has a proximal height along a vertical axis Z at the proximal end of the cage. The cage has a distal height along a vertical axis Z at the distal end of the cage. The first serpentine spring joins the upper support to the lower support along a first serpentine path having a first path length that is at least two times the cage length. The first serpentine path defines the first side of the cage. The second serpentine spring joins the upper support to the lower support along a second serpentine path having a second path length that is at least two times the cage length. The second serpentine path defines the second side of the cage.

In one implementation the at least two independent slider mechanisms include more than two independent slider mechanisms. The more than two independent slider mechanisms can be four independent slider mechanisms that independently adjust a height of the cage for four quadrants of the cage.

### DETAILED DESCRIPTION OF THE PREFERRED EMBODIMENTS

In disclosing an implant and system, certain mutually orthogonal axes X, Y, and Z will be used. The X and Y axes are lateral axes and are generally horizontal. The axis Z is generally vertical. The term “generally” is used because such descriptions are relative to one another rather than absolute. The implant of the present disclosure can be inserted between two sequential vertebrae of a generally vertical spine (mostly but not exactly vertical when a patient is standing). However, it is to be understood that the implant of the present disclosure may find additional applications or implanted orientations. Additionally, the X-axis is generally aligned with a major axis of the implant and can be referred to as the major axis of the implant. The X-axis can also be along the direction the implant is inserted into a patient. The Y-axis can be along a lateral width of the implant. The Z-axis can be along a height of the implant.

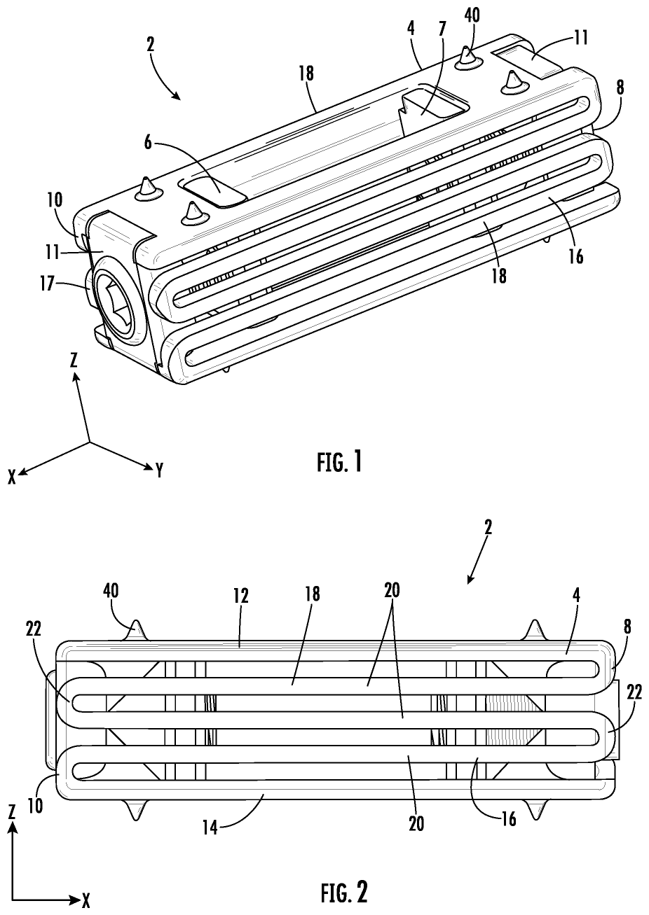

FIG. 1 is an isometric drawing of an implant 2. Implant 2 includes a cage 4 along with slider mechanisms 6, 7 that each at least partially reside within the cage 4. The slider mechanisms 6,7 are configured to alter an outer geometry of the cage 4. Implant 2 (and cage 4) has a proximal end 8 and a distal end 10 that are at opposed or opposite ends of cage 4 with respect to the X-axis. The cage 4 has a cage length along the major axis X of cage 4. The cage 4 has a cage width along lateral axis Y. The cage 4 has a proximal height along the vertical axis Z at the proximal end 8. The cage 4 has a distal height along the vertical axis Z at the distal end 10. The slider mechanisms 6 and 7 are configured for adjusting the proximal and distal heights respectively.

The implant 2 also includes a torsion stabilizer 11 at each of the proximal 8 and distal 10 ends of the cage 4. The torsion stabilizers 11 stabilize the cage 4 to prevent twisting of cage 4 about the major axis X.

In some embodiments, the cage 4 is formed from a titanium alloy. In a particular embodiment, the titanium alloy includes 3-5 percent vanadium, 5-7 percent aluminum, small amounts of other elements such as iron, and the balance or about 88-92% titanium. One such alloy is “Ti-6Al-4V” otherwise referred to as “R56400”. Such a titanium alloy is known for high strength and corrosion resistance. Other possible materials can include other titanium alloys, pure titanium, stainless steel, cobalt-chrome alloys, implantable plastics, and other material suitable for medical or spinal implants. Cage 4 can be formed using additive manufacturing (e.g., three-dimensional printing), subtractive processes such as machining (e.g., mechanical milling and/or electrical discharge machining) or even combinations thereof that are known in the art. One additive manufacture method of forming a titanium alloy cage 4 is by “selective laser melting” of titanium alloy powder using a high powered laser in an inert argon environment as is known in the art. Post-treatments such as heat treating and coatings are also known in the art.

FIG. 2 is a side view of the implant 2. Cage 4 includes an upper support 12 and a lower support 14 that are joined by two serpentine springs 16, 17. The two serpentine springs 16 and 17 define lateral sides 18 of the cage 4 that are on opposite or opposed sides of the cage 4 with respect to the lateral axis Y. In the illustrated embodiment, the serpentine springs 16, 17 each include three linear segments 20 that are joined by two U-shaped bends 22. The serpentine spring 16 (or 17) has a “serpentine” path length from the upper support 12 to the lower support 14 which is greater than the length of cage 4 along the major axis X. The path length is at least two times the length of cage 4 along the major axis X. Preferably, the path length of the serpentine spring 16 (or 17) is approximately equal to three times the length of each linear segment 20 measured along the X axis plus an added path length of the U-shaped bends 22.

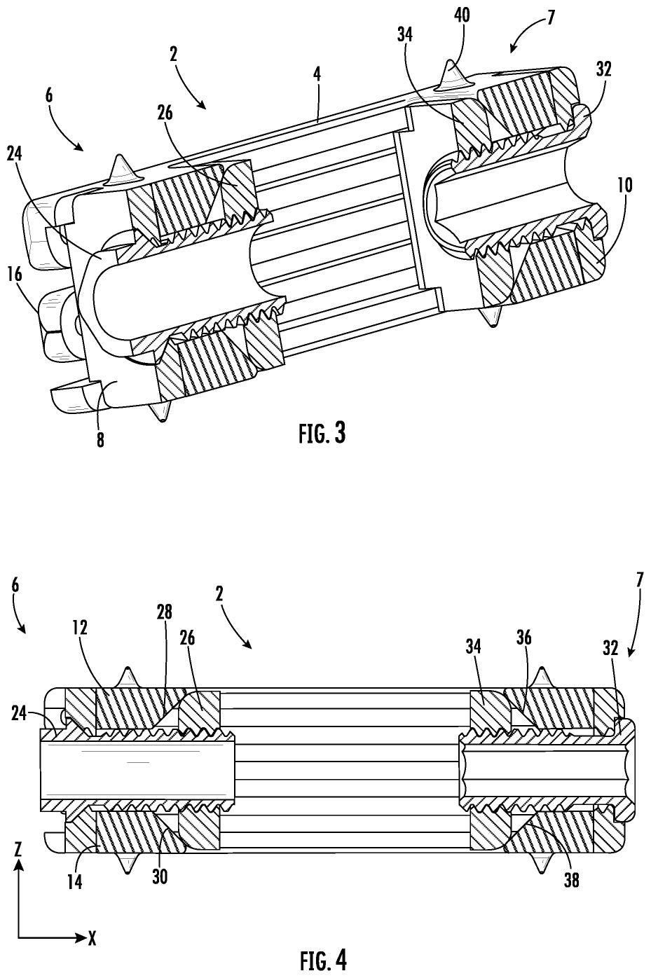

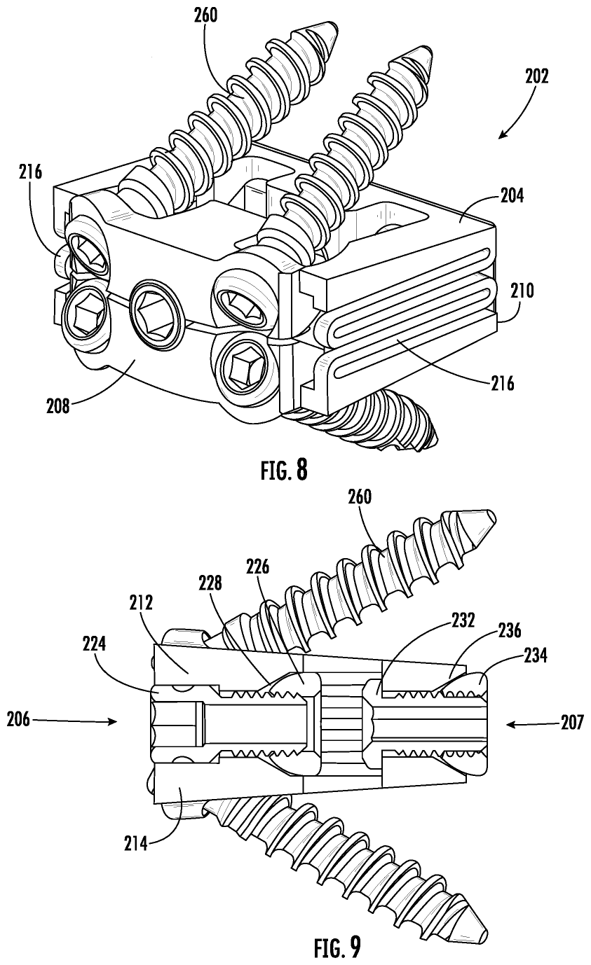

FIG. 3 is an isometric bisectional view of the implant 2 shown in FIG. 1 (cross sectional view bisected through the center of the Y axis in equal parts). FIG. 4 is a similar side vertical cross-sectional view of the implant 2. The slicing plane for each of FIGS. 3 and 4 vertically bisects the implant 2 to illustrate the proximal 6 and distal 7 slider mechanisms. For purposes of referencing the proximal and distal ends (8, 10) of the cage 4 in the Fig.'s, the proximal threaded bolt has an external (outward facing) hexagonal surface and the distal threaded bolt has an internal (inward facing) hexagonal surface, with the exception of FIGS. 8 and 9 wherein the proximal and distal threaded bolts both have internal (inward facing) hexagonal surfaces.

The proximal slider mechanism 6 includes a proximal threaded bolt 24 and a proximal slide 26. The proximal threaded bolt 24 is mechanically restrained along the major axis X with respect to the cage 4. The proximal threaded bolt 24 is threaded to the proximal slide 26. Rotation of the proximal threaded bolt 24 induces a translation of the proximal slide 26 along the major axis X. The proximal slide 26 engages surfaces 28 and 30 of the upper 12 and lower 14 supports respectively. Surface 28 is a lower proximal surface 28 of the upper support 12. Surface 30 is an upper proximal surface 30 of the lower support 14. Surfaces 28 and 30 define a taper.

The engagement of proximal slide 26 against the taper formed by surfaces 28 and 30 causes the upper 12 and lower 14 supports to separate along the vertical axis Z as the proximal slide 26 is translated toward the proximal end 8 of cage 4. Throughout the rotational travel of the proximal threaded bolt 24, the serpentine springs 16 are elastically urging the upper 12 and lower 14 supports together. Therefore, there is generally no hysteresis in the effect of rotating the threaded bolt 24 (except perhaps for a small amount of backlash due to thread engagement tolerances and axial retainment of the threaded bolt by the cage 4 along major axis X) back and forth in two angular directions. Clockwise rotation of threaded bolt 24 therefore increases the proximal height of the cage 4 and counterclockwise rotation of the threaded bolt 24 decreases the proximal height of the cage 4.

The distal slider mechanism 7 includes a distal threaded bolt 32 and a distal slide 34. Due to close similarity of operation, the discussion supra for operation of the proximal slider mechanism 6 applies to the distal slide mechanism 7 and vice-versa. The distal threaded bolt 32 is restrained along the major axis X and is threadedly received into the distal slide 34. As the distal threaded bolt 32 is rotated, the effect is to translate the distal slide 34 along X which in turn engages a taper defined by lower distal surface 36 and upper distal surface 38 of the upper 12 and lower 14 supports respectively. Clockwise rotation of threaded bolt 32 therefore increases the distal height of the cage 4 and counterclockwise rotation of the threaded bolt 32 decreases the distal height of the cage 4.

The proximal slider mechanism 6 has a proximal 8 height range of adjustment. The distal slider mechanism 7 has a distal 10 height range of adjustment. The first 16 and second 17 serpentine springs are each configured to remain within an elastic limit throughout the proximal height range of adjustment and the distal height range of adjustment. During the insertion of implant 2 into a patient the proximal 8 and distal 10 height of the cage 4 is increased to provide support between sequential bone segments or vertebrae. Because the serpentine springs 16, 17 remain within elastic limits, the slider mechanisms 6, 7 can later be adjusted to decrease the proximal 8 and distal 10 height of the cage 4 because of a restoring spring force of serpentine springs 16, 17 that urge the proximal 8 and distal 10 ends of the cage toward a more compact or decreased height condition.

The upper 12 and lower 14 supports include tapered and pointed locking features 40 for locking or restraining the cage 4 against the vertebrae or bone segments. In the illustrated embodiment four locking features 40 extend and taper upward from the upper support 12 and four locking features 40 extend and taper downward from the lower support 14.

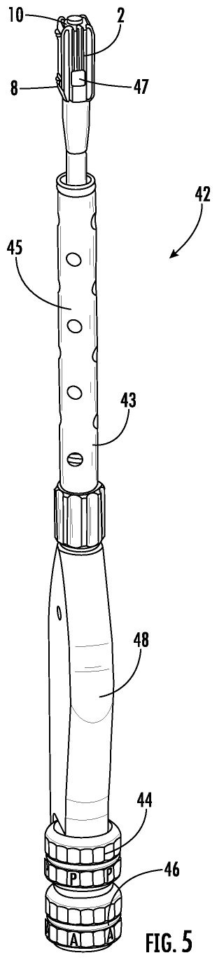

FIG. 5 is an isometric view of a system 42 that includes the implant 2 mounted to an insertion instrument 43 for insertion into a patient. The insertion instrument 43 includes two rotatable drives including a proximal drive 44 and distal drive 46. When the proximal drive 44 is rotated clockwise relative to handle 48, the effect is to vertically expand the proximal end 8 of the cage 4. When the distal drive 46 is rotated clockwise relative to handle 48, the effect is to vertically expand the distal end 10 of the cage 4.

The insertion instrument 43 also includes a sleeve 45 coupled to jaws of a clamp 47. Clockwise rotation of sleeve 45 relative to handle 48 tightens clamp 47 upon the cage 4. Counterclockwise rotation of sleeve 45 relative to handle 48 relaxes and releases clamp 47 from cage 4.

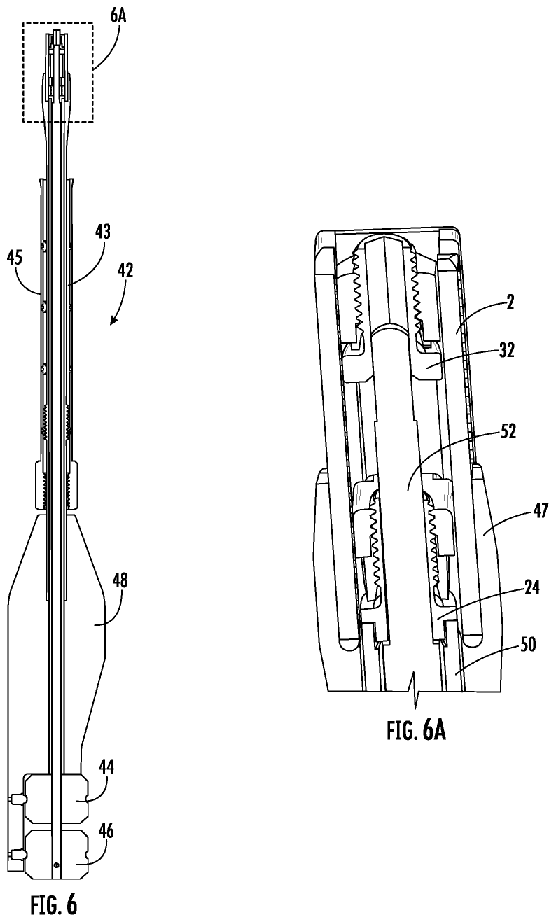

FIG. 6 is a cross-sectional view of the system 42. FIG. 6A is detail taken from FIG. 6. When implant 2 is placed onto the insertion instrument 43, the drives 44 and 46 are coupled to the threaded bolts 24 and 32 respectively. The drives 44 and 46 include coaxial driver heads 50, 52. The proximal drive 44 includes outer driver head 50 that is configured to engage an outer hexagonal surface of the proximal threaded bolt 24. The distal drive 46 includes inner driver head 52 that is configured to engage an inner hexagonal surface of the distal threaded bolt 32.

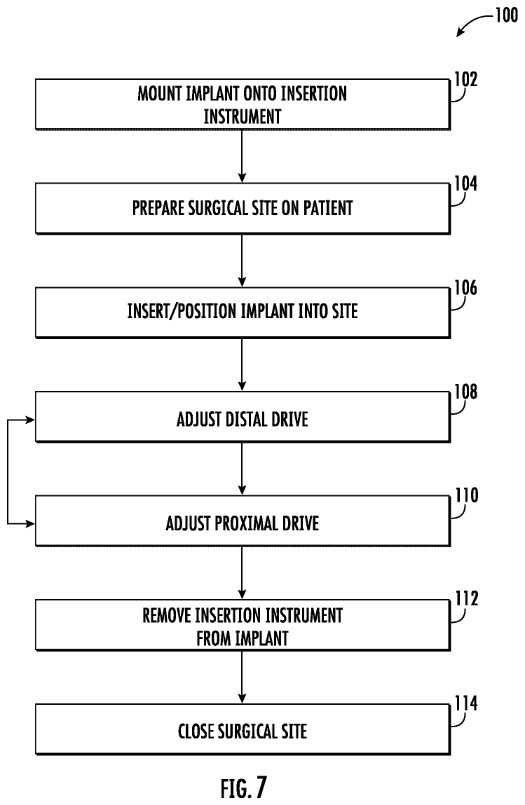

FIG. 7 is a flowchart that depicts a method 100 for implanting the implant 2 between vertebrae of a patient. According to 102, the implant 2 is mounted upon the insertion instrument 43. This includes coupling drives 44 and 46 to threaded bolts 24 and 32 respectively. This also includes rotating sleeve 45 clockwise relative to handle 48 to close and tighten clamp 47 upon cage 4.

According to 104, a surgical site for receiving the implant 2 is prepared. According to 106, the insertion instrument 43 is used to position the implant 2 within the surgical site. According to 108 and 110, the distal 46 and proximal 44 drives are rotated relative to the handle 48 to vertically expand the distal 10 and proximal 8 ends of the cage 4 and to lock the cage 4 in place at the surgical site. As a note, the order of steps 108 and 110 can occur in any order and even repeated until the cage 4 is locked in place. This is indicated by a double arrow that connects steps 108 and 110.

According to 112, the insertion instrument 43 is removed from the implant 2. As part of step 112, the sleeve 45 is rotated counterclockwise to release the clamp 47 from the cage 4. Finally, according to 114, the surgical site is closed.

In the embodiment described with respect to FIGS. 1-7 particular directions were described including clockwise rotations to tighten the clamp 47 and to vertically expand cage 4. In other embodiments, counterclockwise rotations can tighten the clamp 47 and/or to vertically expand cage 4. Thus, all such variations of right and left handed threads and/or angled taper geometries are within the scope of the present disclosure.

FIGS. 8 and 9 are isometric and side section views of a second embodiment of an implant 202. Implant 202 includes screws 260 for attachment of the implant to vertebrae. Otherwise element number comparison between second implant embodiment 202 and implant 2 have similar functions when the number 200 is added to the elements described for FIGS. 1-7.

Implant 202 includes cage 204, proximal 206 and distal 207 slider mechanisms, proximal 208 and distal 210 ends of implant 202, upper 212 and lower 214 supports, first 216 and second 217 serpentine springs, proximal 224 and distal 232 threaded bolts, proximal 226 and distal 234 slides, and proximal 228 and distal 236 surfaces engaged by the slides 226 and 234. Thus, clockwise/counterclockwise rotation of proximal 224 and distal 232 threaded bolts vertically expand/contract proximal 208 and distal 210 ends of implant 202 respectively.

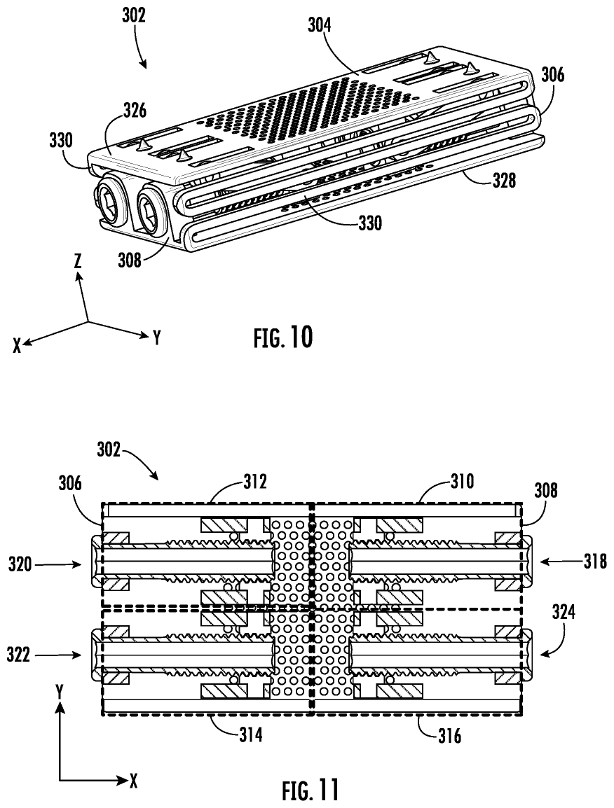

FIGS. 10-11 are isometric and top section views depicting a third embodiment of an implant 302. While implant 302 has some features in common with implant 2, there are also some notable differences. The two differences include (1) an ability to adjust a vertical height independently at four quadrants in X and Y, and (2) a slider mechanism that adjusts height by an action of a linkage. Certain other aspects or options such as the cage material and serpentine springs discussed supra would be very similar.

Implant 302 includes an outer cage 304, a proximal end 306, and a distal end 308. Cage 304 has a cage length along a major axis X between the proximal end 306 and the distal end 308. Cage 304 has a cage width along a lateral axis Y. Cage 304 has four rectangular quadrants with respect to the X and Y axes (see FIG. 11 in particular) including a first quadrant 310, a second quadrant 312, a third quadrant 314, and a fourth quadrant 316. Cage 304 has an adjustable height along vertical axis Z at each of the four quadrants. The adjustable height can be defined in various ways including an average height for the quadrant or a height at a central point of the quadrant (taken as the center in X and Y of a rectangular quadrant).

To independently adjust the quadrant heights, the implant 302 includes a slider mechanism for each quadrant including a first slider mechanism 318 for adjusting the height of first quadrant 310, a second slider mechanism 320 for adjusting the height of second quadrant 312, a third slider mechanism 322 for adjusting the height of third quadrant 314, and a fourth slider mechanism 324 for adjusting the height of fourth quadrant 316.

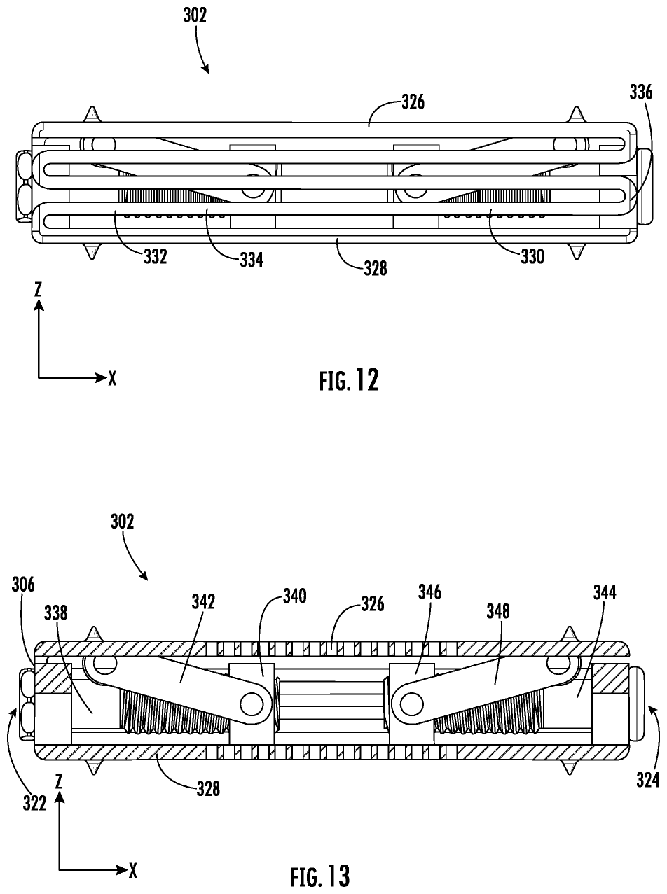

FIG. 12 is a side view of implant 302. Cage 304 includes an upper support 326 joined to a lower support 328 by two serpentine springs 330 (that are on opposite or opposing sides 332 of the cage 304 with respect to the lateral axis Y. In the illustrated embodiment, the serpentine springs 330 each include three linear segments 334 that are joined by two U-shaped bends 336. Each serpentine spring 330 has a serpentine path length that is greater than the cage 304 length along the major axis X of the cage 304. The serpentine path length can be at least or greater than two times the cage 304 length along the major axis X of the cage 304. In the illustrated embodiment, the path length of each serpentine spring 330 is preferably approximately equal to three times the length of each linear segment 334 measured along the X axis plus an added length path length of the U-shaped bends 336.

FIG. 13 is a vertical side cross-sectional view of the implant 302. The slicing plane bisects slider mechanisms 322 and 324 (see FIG. 11). The slider mechanism 322 includes a proximal threaded bolt 338, a proximal slide 340, and a linkage 342. The proximal threaded bolt 338 is mechanically restrained along the major axis X with respect to the cage 304. The proximal threaded bolt 338 is threaded to the proximal slide 340. Rotation of the proximal threaded bolt 338 induces a translation of the proximal slide 340 with respect to the major axis X. The linkage 342 is rotatively coupled to the proximal slide 340 and the upper support 326. A translation of the slide 340 in the −X direction toward the proximal end 306 of the cage 304 will cause the linkage to rotate and push up on the upper support 12 and to increase a height of the third quadrant 314 of the cage 304. Translation of the slide 340 in the +X direction will decrease a height of the third quadrant 314 of the cage 304 to its initial resting position in a like manner.

The slider mechanism 320 has the same parts, function, and mechanical action as the slider mechanism 322. Rotation of a proximal threaded bolt 338 will adjust the height of the second quadrant 312 in a like manner.

The slider mechanism 324 is coaxially aligned with the slider mechanism 322 and includes a distal threaded bolt 344, a distal slide 346, and a linkage 348. The slider mechanism 324 operates in a manner that is similar to that described with respect to the slider mechanism 322 for adjusting the height of the fourth quadrant 316.

The slider mechanism 318 has the same parts, function, and mechanical action as the slider mechanism 324. Rotation of a distal threaded bolt 344 will adjust the height of the first quadrant 310 in a likewise manner. The slider mechanism 318 is coaxially aligned with the slider mechanism 320.

For each pair of coaxially aligned slider mechanisms (318/320 or 322/324) the bolts are coaxial meaning that for each pair, the proximal threaded bolt 338 is coaxial with the distal threaded bolt 344. This allows an insertion instrument that is similar to insertion instrument 43 to be used to place and lock the implant 302 into a surgical site in a manner similar to that described with respect to FIGS. 5, 6, 6A, and 7. In one embodiment, the insertion instrument 43 is used to adjust one pair at a time for coaxially aligned slider mechanisms (318/320 or 322/324). In another embodiment, an insertion mechanism could be used that allows simultaneous adjustment of all four quadrants of the implant 302.

The specific embodiments and applications thereof described above are for illustrative purposes only and do not preclude modifications and variations encompassed by the scope of the following claims. As indicated earlier, clockwise expansion of a cage and counterclockwise contraction can be interchanged with counterclockwise expansion and clockwise contraction for any slider mechanism without departing from the scope of the inventive scope.

What is claimed:

1. An implant comprising:
a cage having cage length along a major axis of the cage between a proximal end and a distal end of the cage, a cage width along a lateral axis of the cage, a proximal height along a vertical axis of the cage at the proximal end of the cage, and a distal height along the vertical axis of the cage at the distal end of the cage, the cage including:
an upper support;
a lower support;
a first serpentine spring joining the upper support to the lower support along a first path having a first path length that is greater than the cage length, the first path generally lying along a first side of the cage; and
a second serpentine spring joining the upper support to the lower support along a second path having a second path length that is greater than the cage length, the second path generally lying along a second side of the cage, a lateral distance between the first side of the cage and the second side of the cage defines the cage width;
a proximal slider mechanism configured to adjust the proximal height of the cage; and
a distal slider mechanism configured to adjust the distal height of the cage.

2. The implant of claim 1 wherein proximal slider mechanism has a proximal height range of adjustment, the distal slider mechanism has a distal height range of adjustment, the first and second serpentine springs are each configured to remain within an elastic strain limit throughout the proximal height range of adjustment and the distal height range of adjustment.

3. The implant of claim 1 wherein the first and second path lengths are each at least two times the cage length.

4. The implant of claim 1 wherein first and second serpentine springs each define two U-shaped bends that connect three linear segments, each linear segment defines a length greater than 80% of the cage length.

5. The implant of claim 1 wherein the proximal slider mechanism includes a proximal threaded bolt that is threaded to a proximal slide, rotating the proximal threaded bolt translates the proximal slide along the major axis.

6. The implant of claim 5 wherein the proximal slider mechanism slidingly engages a surface of the cage to increase or decrease a distance between the upper support and the lower support at the proximal end of the cage.

7. The implant of claim 5 further comprising a linkage coupled between the proximal slide and the upper support, translating the slide rotates the linkage to increase or decrease a distance between the upper support and the lower support at the proximal end of the cage.

8. The implant of claim 1 wherein the distal slider mechanism includes a distal threaded bolt that is threaded to a distal slide, rotating the distal threaded bolt translates the distal slide along the major axis to increase or decrease a distance between the upper and lower support at the distal end of the cage.

9. A system including the implant of claim 1 and further comprising an insertion instrument that is configured to be simultaneously coupled to the proximal and distal slider mechanisms to allow independent and simultaneous adjustment of the vertical distance between the upper and lower supports at both the proximal and distal ends with a single coupling of the insertion instrument to the implant.

10. The implant of claim 1 further including a plurality of tapered locking features that extend vertically upward and downward from the upper and lower supports respectively.

11. The implant of claim 1 further comprising a plurality of bone screws that extend from the upper and lower supports.

12. The implant of claim 1 wherein the proximal slider mechanism includes four slider mechanisms configured to independently adjust a height of the cage for four quadrants of the cage.

13. A method of inserting and adjusting the implant of claim 1 into a patient comprising:
coupling the implant upon an insertion instrument;
manipulating the insertion instrument to position the implant within a surgical site;
operating proximal and distal drives to independently adjust the proximal and distal heights of the cage; and
decoupling the implant from the insertion instrument.

14. The method of claim 13 wherein the insertion instrument includes a sleeve coupled to a clamp, coupling the implant includes rotating the sleeve to close the clamp over the implant.

15. The method of claim 13 wherein operating the proximal and distal drives includes independently rotating the proximal and distal drives.

16. An implant comprising:
a cage having cage length along a major axis of the cage between a proximal end and a distal end of the cage, a cage width along a lateral axis of the cage, a proximal height along a vertical axis of the cage at the proximal end of the cage, and a distal height along the vertical axis of the cage at the distal end of the cage, the cage including:
an upper support;
a lower support;
a first serpentine spring joining the upper support to the lower support along a first path having a first path length that is at least two times the cage length, the first path defining a first side of the cage; and
a second serpentine spring joining the upper support to the lower support along a second path having a second path length that is at least two times the cage length, the second path defining a second side of the cage;
at least two independent slider mechanisms configured to at least adjust the proximal height and the distal height of the cage.

17. The implant of claim 16 wherein the at least two independent slider mechanisms include more than two independent slider mechanisms.

18. The implant of claim 16 wherein the upper support has a lower surface, the lower support has an upper surface, the upper and lower surfaces defining a taper, the at least two independent slider mechanisms include a threaded bolt coupled to a slide, rotating the threaded bolt causes sliding engagement between the slide and the taper defined by the upper and lower surfaces to adjust the proximal and/or the distal height.

19. The implant of claim 16 wherein the at least two independent slider mechanisms include a threaded bolt coupled to a slide and further comprising a linkage coupled between the slide and the upper support, rotating the threaded bolt causes translation of the slide and consequent rotation of the linkage, rotating of the linkage adjusts the proximal and the distal height.

20. An implant comprising:
a cage having cage length along a major axis of the cage between a proximal end and a distal end of the cage, a cage width along a lateral axis of the cage, a proximal height along a vertical axis of the cage at the proximal end of the cage, and a distal height along the vertical axis of the cage at the distal end of the cage, the cage including:
an upper support;
a lower support;
a first serpentine spring joining the upper support to the lower support along a first path having a first path length that is greater than the cage length, the first path defining a first side of the cage; and
a second serpentine spring joining the upper support to the lower support along a second path having a second path length that greater than the cage length, the second path defining a second side of the cage;
at least two slider mechanisms configured to at least independently adjust the proximal height and the distal height of the cage, the at least two slider mechanisms each have a maximum range of travel, the first serpentine spring and the second serpentine spring remain within an elastic strain limit throughout the maximum range of travel of the proximal and distal slider mechanisms.
